(19) United States
(12) Patent Application Publication
(10) Pub. No.: US 2022/0400958 A1
(43) Pub. Date: Dec. 22, 2022

(54) USER MOBILE DEVICE INPUT INTERFACE WITH INTEGRATED BLOOD PRESSURE DETECTION

(71) Applicant: SHENZHEN GOODIX TECHNOLOGY CO., LTD., Shenzhen (CN)

(72) Inventors: Yi He, San Diego, CA (US); Bo Pi, San Diego, CA (US)

(21) Appl. No.: 17/353,819

(22) Filed: Jun. 22, 2021

(51) Int. Cl. A61B 5/022; A61B 5/026; A61B 5/00

(52) U.S. Cl. CPC A61B 5/022; A61B 5/026; A61B 5/748; A61B 5/6898; A61B 5/742; A61B 5/7405; A61B 5/7455; G06F 2203/04105; G06K 9/0004

(57) ABSTRACT

Techniques are described for integrating blood pressure measurement (BPM) into a portable electronic device. For example, an input interface of the device includes an integrated force sensor. Human-discernable feedback is output to the user, while using the force sensor to monitor fingertip pressure being applied by the user on the input interface, to guide the user into a first condition in which capillary fingertip blood flow (CFBF) is occluded. The human-discernable feedback is then output to the user, while continuing to use the force sensor to monitor the fingertip pressure, to guide the user into one or more subsequent conditions that allow non-occluded CFBF signals to be sensed by one or more sensors (e.g., the force sensor, an optical fingerprint sensor, etc.). The sensed non-occluded CFBF signals can be used to generate one or more CFBF-based BPM readings for the user (e.g., which can be calibrated to arterial BPM).

### TECHNICAL FIELD

This disclosure relates to mobile device input interfaces, and, more particularly, to integrating blood pressure detection in user mobile device interfaces using integrated input interfaces with dynamic force sensor feedback.

### BACKGROUND

Various sensors can be implemented in electronic devices or systems to provide certain desired functions. Some sensors detect static types of user information, such as fingerprints, iris patterns, etc. Other sensors detect dynamic types of user information, such as body temperature, pulse, etc. The various types of sensors can be used for many different purposes. In some cases, such sensors help enable user authentication, for example, to protect personal data and/or prevent unauthorized access to user devices. In other cases, such sensors can help monitor changes in physical and/or mental state of a user, such as for fitness tracking, biofeedback, etc. To support these and other purposes, various types of sensors can be in communication with, or even integrated with, devices and systems, such as portable or mobile computing devices (e.g., laptops, tablets, smartphones), gaming systems, data storage systems, information management systems, large-scale computer-controlled systems, and/or other computational environments.

As one set of examples, authentication on an electronic device or system can be carried out through one or multiple forms of biometric identifiers, which can be used alone or in addition to conventional password authentication methods. A popular form of biometric identifiers is a person's fingerprint pattern. A fingerprint sensor can be built into the electronic device to read a user's fingerprint pattern so that the device can only be unlocked by an authorized user of the device through authentication of the authorized user's fingerprint pattern. Another example of sensors for electronic devices or systems is a biomedical sensor that detects a biological property of a user, e.g., a property of a user's blood, the heartbeat, in wearable devices like wrist band devices or watches. In general, different sensors can be provided in electronic devices to achieve different sensing operations and functions. Such sensing operations and functions can be used as stand-alone authentication methods and/or in combination with one or more other authentication methods, such as a password authentication, or the like.

Different types of sensors have been integrated in different ways, and to different extents, with mobile electronic devices. For example, many modern smart phones have integrated accelerometers, cameras, and even fingerprint sensors. However, each such sensor integration has involved careful consideration of and compliance with technical, design, and other constraints, such as imposed limits on physical space, power, heat generation, cost, external access (e.g., for sensors relying on physical contact or optical access), interference with interface elements (e.g., a display screen, buttons, etc.), etc.

### SUMMARY

Embodiments provide systems and methods for integrating blood pressure measurement (BPM) into a portable electronic device. For example, an input interface of the device includes an integrated force sensor. Human-discernable feedback is output to the user, while using the force sensor to monitor fingertip pressure being applied by the user on the input interface, to guide the user into a first condition in which capillary fingertip blood flow (CFBF) is occluded. The human-discernable feedback is then output to the user, while continuing to use the force sensor to monitor the fingertip pressure, to guide the user into one or more subsequent conditions that allow non-occluded CFBF signals to be sensed by one or more sensors (e.g., the force sensor, an optical fingerprint sensor, etc.). The sensed non-occluded CFBF signals can be used to generate one or more CFBF-based BPM readings for the user (e.g., which can be calibrated to arterial BPM).

According to one set of embodiments, a system is provided for measuring blood pressure of a user. The system includes one or more processors configured to communicate with a device input interface of the portable electronic device and at least one device output interface of a portable electronic device, the device input interface having a set of sensors integrated therewith including a force sensor; and a non-transient, processor-readable memory having instructions stored thereon, which, when executed, cause the one or more processors to perform steps. The steps include: first sensing capillary fingertip blood flow (CFBF) signals by the set of sensors for a fingertip by which the user is presently applying fingertip pressure to the device input interface, the CFBF signals corresponding to heartbeat signals of the user; outputting, concurrent with the first sensing, first human-discernable feedback via the at least one device output interface, based on monitoring the fingertip pressure by the force sensor, to guide the user to increase the fingertip pressure until the first sensing detects occlusion of the CFBF; and obtaining a non-occluded blood pressure measurement (BPM), responsive to the detecting the occlusion of the CFBF, by: second sensing the CFBF signals by the set of sensors; and outputting, concurrent with the second sensing, second human-discernable feedback via the at least one device output interface, based on monitoring the fingertip pressure by the force sensor, to guide the user to reduce the fingertip pressure until the second sensing detects a non-occluded CFBF, such that the non-occluded BPM is obtained based on the CFBF signals as sensed upon the detecting the non-occluded CFBF.

According to another set of embodiments, a method is provided for measuring blood pressure of a user by a portable electronic device having a device input interface and at least one device output interface, the device input interface having a set of sensors integrated therewith including a force sensor. The method includes: first sensing capillary fingertip blood flow (CFBF) signals by the set of sensors for a fingertip by which the user is presently applying fingertip pressure to the device input interface, the CFBF signals corresponding to heartbeat signals of the user; outputting, concurrent with the first sensing, first human-discernable feedback via the at least one device output interface, based on monitoring the fingertip pressure by the force sensor, to guide the user to increase the fingertip pressure until the first sensing detects occlusion of the CFBF; and obtaining a non-occluded blood pressure measurement (BPM), responsive to the detecting the occlusion of the CFBF, by: second sensing the CFBF signals by the set of sensors; and outputting, concurrent with the second sensing, second human-discernable feedback via the at least one device output interface, based on monitoring the fingertip pressure by the force sensor, to guide the user to reduce the fingertip pressure until the second sensing detects a non-occluded CFBF, such that the non-occluded BPM is obtained based on the CFBF signals as sensed upon the detecting the non-occluded CFBF. In some embodiments, the method also includes outputting the non-occluded BPM via the at least one device output interface.

### BRIEF DESCRIPTION OF THE DRAWINGS

The accompanying drawings, referred to herein and constituting a part hereof, illustrate embodiments of the disclosure. The drawings together with the description serve to explain the principles of the invention.

FIG. 1 shows a block diagram of a portable electronic device, according to various embodiments described herein.

FIGS. 2A and 2B show top and side views, respectively, of a first portable electronic device implemented as a smart phone with a discrete optical fingerprint sensor package.

FIGS. 3A and 3B show top and side views, respectively, of a second portable electronic device implemented as a smart phone with an under-display optical fingerprint sensor package.

FIG. 4 shows a diagram of an example calibration environment, according to various embodiments.

FIG. 5 shows a flow diagram of an illustrative method for measuring blood pressure of a user by a portable electronic device, according to various embodiments.

In the appended figures, similar components and/or features can have the same reference label. Further, various components of the same type can be distinguished by following the reference label by a second label that distinguishes among the similar components. If only the first reference label is used in the specification, the description is applicable to any one of the similar components having the same first reference label irrespective of the second reference label.

### DETAILED DESCRIPTION

In the following description, numerous specific details are provided for a thorough understanding of the present invention. However, it should be appreciated by those of skill in the art that the present invention may be realized without one or more of these details. In other examples, features and techniques known in the art will not be described for purposes of brevity.

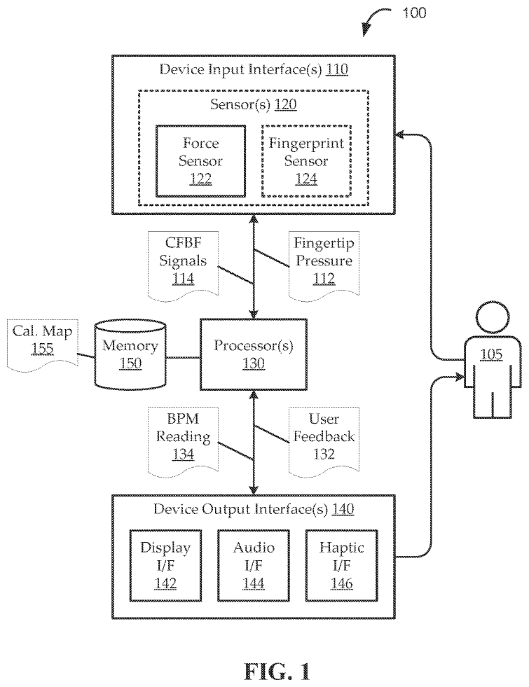

Turning to FIG. 1, a block diagram is shown of a portable electronic device 100, according to various embodiments described herein. A user 105 is shown interacting with the portable electronic device 100 for context. In some implementations, the portable electronic device 100 is a mobile device that the user 105 may typically have on their person, such as a smart phone, smart wearable device (e.g., smart watch, fitness tracker, etc.). In other implementations, the portable electronic device 100 is a laptop computer, a tablet computer, an electronic reader, or the like. As illustrated, the portable electronic device 100 includes one or more device input interfaces 110, one or more device output interfaces 140, one or more processors 130, and a memory 150 (e.g., including one or more storage devices). Such a portable electronic device 100 may also include other components that are not shown, such as one or more wired or wireless network interfaces (e.g., for communicating with wireless fidelity (WiFi), Bluetooth, ZigBee, cellular, satellite, Ethernet, cable, and/or any other suitable wired or wireless communication network), one or more peripheral interfaces (e.g., a headphone jack, display port, etc.), etc.

As described herein, embodiments of the portable electronic device 100 include integrated blood pressure detection features. Some such features relate to the blood pressure detection itself, such as measuring and outputting a current blood pressure measurement for the user 105, detecting a significant change in blood pressure, etc. Other such features are indirectly related to the blood pressure detection, such as using the blood pressure detection for spoof detection (e.g., determining that the blood pressure is characteristic of a live human finger, rather than a spoof). In general, the portable electronic device 100 is assumed to have one or more primary functions that are not related to the blood pressure detection. For example, in a smart phone implementation, the smart phone is designed to provide a number of primary functions, such as telephony, data communications, and media playback; and the integrated blood pressure detection features are secondary (e.g., enhancements or additions to primary functions of the smart phone).

Embodiments of the one or more processors 130 can include a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction set computer (RISC) processor, a complex instruction set computer (CISC) processor, a microprocessor, or the like, or any combination thereof. The one or more processors 130 can be in communication with the memory 150, which can include at least non-transient storage for providing processor-readable instructions to the processor(s) 130 and for storing various types of data to support features described herein. In some embodiments, the memory 150 is all local storage (e.g., one or more solid-state drives, hard disk drives, registers, etc.) of the portable electronic device 100. Additionally or alternatively, embodiments of the memory 150 can include remote storage (e.g., a remote server), distributed storage (e.g., cloud-based storage), or other non-local storage.

Embodiments of the processor(s) 130 communicate with the device input interfaces 110 and the device output interfaces 140, such as in accordance with executing instructions stored in the memory 150. The processor(s) 130 can receive and process data from the device input interfaces 110, and the processor(s) 130 can generate and communicate data to the device output interfaces 140. Some processor 130 interactions are interface-driven, and others are processor-driven. As an example of a processor-driven interaction, one of the processors 130 can direct one of the device input interfaces 110 to obtain data and to send the data to the processor 130 in response to the direction. As an example of an interface-driven interaction, a device input interface 110 can be in an always-on (e.g., standby) mode waiting to detect an input, and detection of an input triggers an interrupt that drives the processor(s) 130 to take responsive action.

Embodiments of the device output interfaces 140 can include any suitable components or collections of components by which human-discernable feedback can be provided to the user 105. In some implementations, the device output interfaces 140 include one or more display interfaces 142 by which to provide visual output to the user 105. For example, a portable electronic device 100 may include one or more light emitting diode (LED) displays, organic LED (OLED) displays, liquid crystal displays (LCDs), plasma displays, LED indicators, and/or other displays that can output text, graphics, and/or any other visual information to the user 105. In some implementations, the device output interfaces 140 include one or more audio interfaces 144 by which to provide audible output to the user 105. For example, a portable electronic device 100 may include one or more speakers, buzzers, or other audio transducers that can output audible tones, recorded and/or synthesized speech, music, and/or any other audible information to the user 105. In some implementations, the device output interfaces 140 include one or more haptic interfaces 146 by which to provide haptic output to the user 105. For example, a portable electronic device 100 may include one or more vibration motors, or other component that can produce a particular output felt by the user 105.

Embodiments of the device input interfaces 110 can include any suitable components or collections of components that provide a physical interface between the user 105 and features of the portable electronic device 100 and have a set of (i.e., one or more) sensors 120 integrated therewith. For example, the device input interfaces 110 can include one or more physical buttons, physical switches, touchscreens, structural features of a chassis or housing, etc. At least one of the sensors 120 integrated with at least one of the device input interfaces 110 is a force sensor 122 that detects a magnitude of compression force being exerted on the sensor. The force sensor 122 can be implemented as a force micro-sensor load cell (or cells) having foil strain gauges bonded to a small flexure body. Application of compression force causes the flexure body to deform slightly, thereby changing the shape and electrical properties of the strain gauges in an electrically detectable manner. For example, deformation of the strain gauges causes a change in resistance, which can be converted to a change in voltage (e.g., via a Wheatstone bridge, or the like) that is proportional to the compression force being exerted on the flexure. In some embodiments, the sensors 120 also include one or more fingerprint sensors 124, such as one or more optical fingerprint sensors. In some embodiments, the sensors 120 include any other suitable type of sensor, such as an ultrasound sensor, photo-detection sensor, camera, temperature sensor, accelerometer, etc.

The force sensor 122 (e.g., and one or more other sensors 120) can be integrated with the device input interfaces 110 in any suitable manner, such that the user 105 can apply pressure (compression force) to the force sensor 122 in connection with the user 105 interfacing with the device input interface 110. For example, pressing on a button or a touchscreen interface applies pressure to the force sensor 122. In some implementations, the same device input interface 110 with which the force sensor 122 has other integrated sensors 120. For example, a same physical button has an integrated force sensor 122 and optical fingerprint sensor 124 (e.g., and/or ultrasonic sensor, temperature sensor, humidity sensor, etc.). In some implementations, one or more of the device input interfaces 110 can also be integrated with one or more of the device output interfaces 140. For example, the same physical button both receives inputs as a button, receives data via integrated sensors 120, and provides user-discernable feedback via an indicator light (display interface 142), a vibration motor (haptic interface 146), or some other device output interface 140.

As described above, the processor(s) 130 are configured to communicate with at least one of the device output interfaces 140 and at least one of the device input interfaces 110 that has the force sensor 122 integrated therewith. For the sake of clarity, the at least one of the device input interfaces 110 that has the force sensor 122 integrated therewith will be referred to as “the device input interface 110.” The processor(s) 130 can obtain a blood pressure measurement (BPM) from the device input interface 110 in at least two phases: an “occluded” detection phase; and at least one “non-occluded” collection phase. In each phase, the processor(s) 130 can direct at least one device output interface 140 to provide human-discernable feedback to the user 105 to guide the user 105 into applying an appropriate amount of fingertip pressure 112 on the device input interface 110 (based on monitoring the fingertip pressure using the force sensor 122), so that the processor(s) 130 can obtain an appropriate BPM reading 134.

Collection of the BPM by the processor(s) 130 can begin in response to any suitable trigger indicating to initiate collection of a BPM. In some implementations, the user 105 explicitly indicates a desire to initiate collection of the BPM, such as by opening or interacting with an application of the portable electronic device 100 that is associated with BPM collection (e.g., a BPM monitoring application, a fitness tracking application, etc.). In other implementations, collection of the BPM is initiated without explicit direction from the user 105, such as by automatically triggering collection of the user's 105 BPM as part of biometric authentication of the user 105, etc. Whether based on explicit or implicit direction from the user 105, collection of the user's 105 BPM can be further triggered by an interaction with the device input interface 110, such as responsive to the user 105 pressing a button of the device input interface 110, triggering an optical fingerprint sensor 124 of the device input interface 110, triggering a proximity sensor of the device input interface 110, triggering a bioelectrical sensor (e.g., a capacitive and/or resistive sensor that electrically detects presence of the user's fingertip) of the device input interface 110, etc.

As described herein, embodiments obtain blood pressure measurements (BPMs) for the user 105 based on detecting capillary fingertip blood flow (CFBF) 114 for a fingertip by which the user 105 is presently applying fingertip pressure 112 to the device input interface 110. The CFBF corresponds to arterial blood flow for the user 105, such that a BPM obtained based on the CFBF corresponds to (e.g., is functionally related to) a BPM obtained based on arterial measurements (e.g., by applying a blood pressure cuff and/or other sphygmomanometer to the brachial artery, or other major artery). For example, a capillary blood pressure measured in a fingertip may be 5-10 times smaller than if measured in a brachial artery (e.g., on the order of 10-20 mmHg). One or more of the set of sensors 120 is used to sense CFBF of the fingertip while the force sensor 122 is used to monitor the fingertip pressure 112, and relevant human-discernable feedback 132 is output to the user (via one or more device output interfaces 140). The CFBF 114 can be sensed by any suitable one or more of the set of sensors 120, such as by detecting blood fluid dynamic forces using the force sensor 122, by detecting optical impacts of blood fluid dynamics in the fingertip (e.g., based on multispectral and/or other optical techniques) using the optical fingerprint sensor 124, by detecting acoustic impacts of blood fluid dynamics in the fingertip using an ultrasound sensor, etc.

In a first phase, the processor(s) 130 can seek to produce an occluded CFBF condition. Embodiments dynamically generate and output human-discernable feedback 132 via at least one device output interface 140 (e.g., visually, audibly, haptically, etc.) based on monitoring the fingertip pressure 112 by the force sensor 122. The human-discernable feedback 132 is used to guide, or otherwise instruct the user 105 to increase the fingertip pressure 112 until CFBF is occluded (e.g., fully occluded). During the outputting and monitoring, the CFBF signals 114 are sensed (e.g., continuously, by periodic sampling, etc.) to detect when the CFBF is occluded. For example, one or more sensors 120 detects that CFBF is no longer being sensed, even though the force sensor 122 is showing that fingertip pressure 112 is being applied to the device input interface 110 (i.e., with such fingertip pressure 112, CFBF signals 114 should be sensed, if not occluded).

In a second phase, the processor(s) 130 obtain a non-occluded BPM responsive to detecting the occlusion of the CFBF in the first phase. Embodiments continue to dynamically generate and output human-discernable feedback 132 via the at least one device output interface, and/or dynamically generate and output additional or alternative human-discernable feedback 132 via the same or a different at least one device output interface 140. Either way, the human-discernable feedback 132 is generated based on the monitoring of the fingertip pressure 112 by the force sensor 122. The human-discernable feedback 132 is used to guide, or otherwise instruct the user 105 to decrease the fingertip pressure 112 until non-occluded CFBF is detected. During the outputting and monitoring, the CFBF signals 114 are sensed (e.g., continuously, by periodic sampling, etc.) to detect when the CFBF is not occluded. For example, one or more sensors 120 detects CFBF signals 114 with reduced fingertip pressure 112 being applied to the device input interface 110 according to the force sensor 122, though none were previously detected (in the first phase). Upon detecting the CFBF is not occluded, the sensed CFBF signals 114 are used by the processor(s) 130 to generate a BPM reading 134.

In some cases, the non-occluded BPM corresponds to a systolic BPM of the user. In other embodiments, the non-occluded BPM corresponds to a diastolic BPM of the user. In other embodiments, multiple non-occluded BPMs are obtained as corresponding to both a systolic BPM and a diastolic BPM of the user. In some such embodiments, a systolic BPM is obtained by the processor(s) 130, responsive to the detecting the occlusion of the CFBF, by guiding the user 105 (using the human-discernable feedback 132 based on monitoring fingertip pressure 112 by the force sensor 122) to gradually decrease fingertip pressure 112 to a level where systolic CFBF occurs as sensed by one or more sensors 120; and a diastolic BPM is obtained, responsive to obtaining the systolic BPM, by guiding the user 105 (using the human-discernable feedback 132 based on monitoring fingertip pressure 112 by the force sensor 122) to further decrease fingertip pressure 112 to a level where there is diastolic CFBF as sensed by one or more sensors 120. In some such embodiments, the processor(s) 130 are configured to commence obtaining the systolic BPM automatically upon the detecting the occlusion of the CFBF; and to commence obtaining the diastolic BPM automatically upon the obtaining the systolic BPM.

The generated BPM reading 134 can be output in any suitable manner. In some implementations, the BPM reading 134 is output to the memory 150. In other implementations, the BPM reading 134 is output via to one or more device output interfaces 140, such as for display via the display interface 142. In other implementations, the BPM reading 134 is output to a peripheral device, a network-connected device, another computational platform, etc. In embodiments for which the BPM reading 134 includes systolic and diastolic BPM information, the outputting the BPM reading 134 can involve outputting one or both of the systolic and diastolic BPMs. In some embodiments, as described herein, the memory 150 can include a calibration mapping 155 that associates non-occluded BPM as based on the CFBF of the user with non-occluded BPM as based on arterial blood flow of the user. For example, a calibration routine is used to generate the calibration mapping 155 based on one or more non-occluded BPMs obtained based on the CFBF of the user and one or more corresponding non-occluded BPMs obtained concurrently based on arterial blood flow of the user. The various measurements can be used to generate the calibration mapping 155 as a lookup table, as a mathematical formulaic relationship, etc. In such embodiments, obtaining the BPM reading 134 can involve converting the non-occluded BPM obtained based on CFBF into an arterial BPM in accordance with the calibration mapping 155. For example, outputting the BPM reading 134 can involve outputting the arterial BPM value(s) in addition to, or instead of, outputting the CFBF-based BPM measurement.

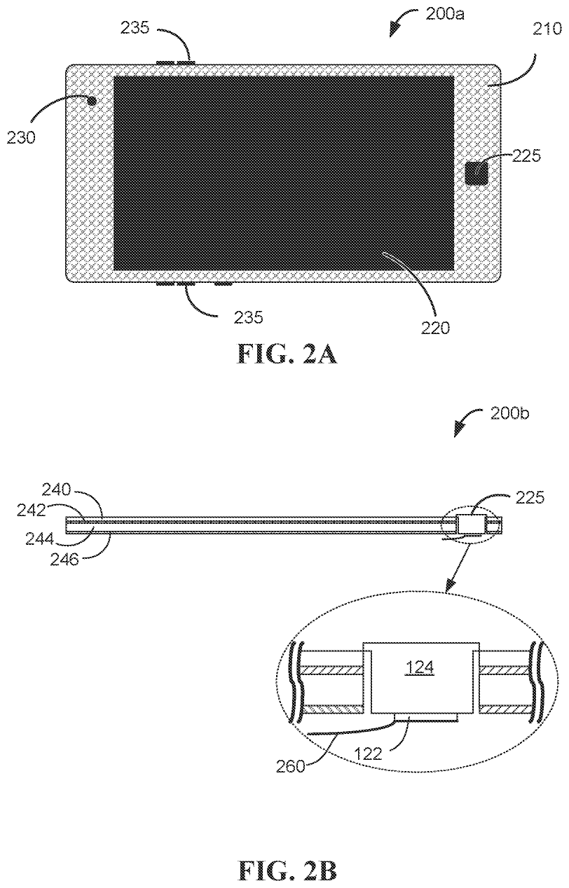

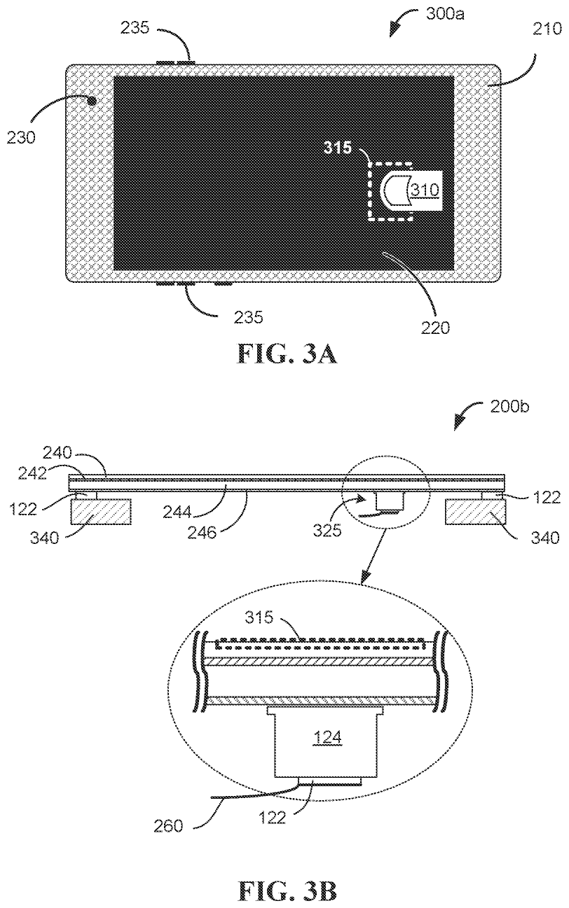

For the sake of illustration, FIGS. 2A-3B show example implementations of portable electronic devices 100 for use with embodiments described herein. FIGS. 2A and 2B show top and side views, respectively, of a first portable electronic device 200 implemented as a smart phone with a discrete optical fingerprint sensor package 225. As illustrated by the top view of the portable electronic device 200 (designated by reference designator 200a in FIG. 2A), embodiments of the portable electronic device 200 include a housing 210 that integrates the discrete optical fingerprint sensor package 225 and other features, such as a display screen 220 (e.g., a touchscreen display), one or more additional sensors (e.g., photo-sensor 230), one or more physical buttons 235, and any other suitable device input interfaces 110 and/or device output interfaces 140.

As illustrated by the side view of the portable electronic device 200 (designated by reference designator 200b in FIG. 2B), the discrete optical fingerprint sensor package 225 can be installed into the housing 210 of the portable electronic device 200. For example, the portable electronic device 200 can include a number of layers to support display and/or other functionality, such as an enhancement cover glass layer 240, a colored material layer 242, a support layer 244, and a conductive pattern layer 246 (e.g., patterns of conductive indium-tin oxide (ITO) to support liquid crystal alignment for a LCD). The discrete optical fingerprint sensor package 225 can be installed in any suitable location, such as in a matched hole of the display 120 (e.g., in a matched hole through the various layers, as illustrated), in a physical button located on the front, side, or back of the portable electronic device 200, in a back structure of the portable electronic device 200, etc.

The discrete optical fingerprint sensor package 225 can be seen more clearly in the zoomed-in region designated by the dashed ellipse. As illustrated, the discrete optical fingerprint sensor package 225 has one or more sensors integrated therein, including a fingerprint sensor 124, the force sensor 122 implemented as a micro-sensor, and a printed circuit board (PCB) 260 (e.g., a flexible PCB). Though not shown, in some implementations, the discrete optical fingerprint sensor package 225 can be configured as a physical button interface and/or can have additional features, sensors, device input interfaces 110, device output interfaces 140, etc. integrated therein. As described herein, the force sensor 122 can be used to detect a magnitude of fingertip pressure being applied on the discrete optical fingerprint sensor package 225.

Some embodiments of fingerprint sensors having pressure sensing capabilities integrated therein are described in U.S. Pat. No. 10,325,142, titled “MULTIFUNCTION FINGERPRINT SENSOR,” filed Apr. 26, 2016; and U.S. Pat. No. 10,635,878, titled “OPTICAL FINGERPRINT SENSOR WITH FORCE SENSING CAPABILITY,” filed Jul. 18, 2017; both of which are incorporated herein by reference in their entirety.

For example, a smart phone is implemented with a “home” button, or the like, that integrates the discrete optical fingerprint sensor package 225 (with the fingerprint sensor 124 and the force sensor 122), such that the home button provides a number of features. Some features use its push-button functionality. For example, by pressing on the button, the user 105 can return to a home screen. Other features use its fingerprint sensor 124 functionality. For example, to unlock the smart phone or provide an application with biometric verification, the user 105 can place a finger on the button, thereby triggering the fingerprint sensor 124 to perform a biometric authentication of the user 105. Other features use its force sensor 122 functionality. For example, as described herein, the force sensor 122 can be used to detect an amount of fingertip pressure being exerted on the button, which can be used to support obtaining of a blood pressure measurement. Other features use combinations of functionalities. As one example, to unlock the smart phone, the user 105 presses on the button, which triggers the fingerprint sensor 124 to obtain biometric fingerprint data and triggers the force sensor 122 to obtain blood flow (e.g., blood pressure, pulse, etc.) data, and the biometric fingerprint and blood flow data are used, together, to perform biometric and liveness authentication of the user 105 to permit or deny access to the smart phone. As another example, the force sensor 122 and the fingerprint sensor 124 work together, as described herein, to obtain a blood pressure measurement. Some embodiments of the discrete optical fingerprint sensor package 225 include additional features, such as one or more device output interfaces 140 integrated therewith. For example, while the user's 105 fingertip is on the button, the user 105 can feel haptic feedback being generated by an integrated haptic interface 146 (e.g., vibrations generated by a vibration motor), see visual feedback being generated by a display interface 142 (e.g., illumination of an indicator lamp), etc.

FIGS. 3A and 3B show top and side views, respectively, of a second portable electronic device 300 implemented as a smart phone with an under-display optical fingerprint sensor package 325. As illustrated by the top view of the portable electronic device 300 (designated by reference designator 200a in FIG. 2A), embodiments of the portable electronic device 300 include a housing 210 that integrates various features, such as a display screen 220 (e.g., a touchscreen display), one or more sensors (e.g., photo-sensor 230), one or more physical buttons 235, and any other suitable device input interfaces 110 and/or device output interfaces 140. Though not explicitly shown in the top view, the portable electronic device 300 also has an integrated under-display optical fingerprint sensor package 325. The positioning, field of view, and other characteristics of the under-display optical fingerprint sensor package 325 define a region of the display screen 220 through which the under-display optical fingerprint sensor package 325 can perform sensing functions, which is illustrated as sensing area 315. For example, the under-display optical fingerprint sensor package 325 can perform fingerprint sensing functions for a fingertip 310 placed in the sensing area 315 of the display screen 220.

As illustrated by the side view of the portable electronic device 300 (designated by reference designator 300b in FIG. 3B), the under-display optical fingerprint sensor package 325 can be installed into the housing 210 of the portable electronic device 300 under an area of the display screen 220. For example, as in FIG. 2B, the portable electronic device 300 can include a number of layers to support display and/or other functionality, such as an enhancement cover glass layer 240, a colored material layer 242, a support layer 244, and a conductive pattern layer 246. The under-display optical fingerprint sensor package 325 can be installed in any suitable location below those layers and can be configured to perform imaging, ultrasound, and/or other sensing functions through those layers. Some examples of under-display implementations of fingerprint sensors that can be adapted to use with some embodiments herein are described in U.S. Pat. No. 10,410,036, titled “UNDER-SCREEN OPTICAL SENSOR MODULE FOR ON-SCREEN FINGERPRINT SENSING,” filed Jan. 31, 2017, which is incorporated herein by reference in its entirety.

The under-display optical fingerprint sensor package 325 can be seen more clearly in the zoomed-in region designated by dashed ellipse. As illustrated, the under-display optical fingerprint sensor package 325 has one or more sensors integrated therein, including a fingerprint sensor 124, the force sensor 122 implemented as a micro-sensor, and a PCB 260. Though not shown, in some implementations, the under-display optical fingerprint sensor package 325 can have additional features, sensors, device input interfaces 110, device output interfaces 140, etc. integrated therein. As described herein, the force sensor 122 can be used to detect a magnitude of fingertip pressure being applied on the display screen 220 in the region of the sensing area 315. Additionally or alternatively to integrating the force sensor 122 under the fingerprint sensor 124, the force sensor 122 can be integrated with the under-display optical fingerprint sensor package 325 by placing one or more force sensors 122 in force communication with the display screen 220. For example, as shown in FIG. 3B, force sensors 122 can be integrated with one or more corners of the display screen 220 so as to detect fingertip pressure on the display screen 220. The fingerprint sensor 124 and one or more force sensors 122 can provide similar features to those described above with reference to FIGS. 2A and 2B.

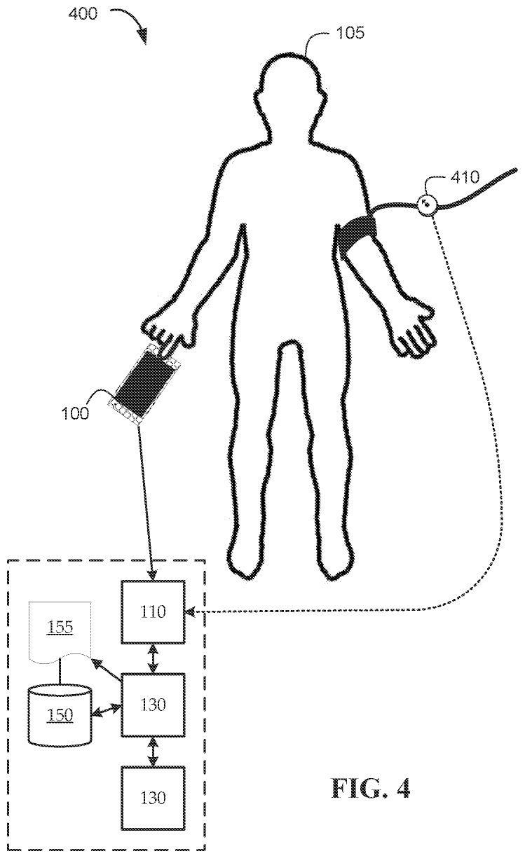

As described herein, some embodiments include calibration from CFBF-based BPMs to arterial BPMs. FIG. 4 shows a diagram of an example calibration environment 400, according to various embodiments. As illustrated, a user 105 is concurrently obtaining CFBF-based BPM readings and arterial BPM readings. The user's 105 finger is on a device input interface 110 of a portable electronic device 100. For example, the user's 105 finger is shown applying pressure to a button interface of a smart phone. Concurrently, a sphygmomanometer 410 is positioned on an artery of the user 105. For example, a blood pressure cuff is shown strapped around the user's 105 brachial artery on the upper arm.

As described with reference to FIG. 1, the user 105 can be guided first into an occluded CFBF condition, and then into one or more non-occluded CFBF conditions, using human-discernable feedback 132 on the portable electronic device 100 based on monitoring of fingertip pressure 112 by a force sensor 122 of the portable electronic device 100, while one or more sensors 120 of the portable electronic device 100 is sensing CFBF signals 114. Such techniques can be used to obtain one or more CFBF-based BPM readings. Concurrently, the sphygmomanometer 410 is used to obtain one or more arterial BPM readings (e.g., corresponding to blood flow through the brachial artery). In some implementations, the sphygmomanometer 410 readings are obtained by the portable electronic device 100 by manual user input via one or more device input interfaces 110 (e.g., by entering the readings into an application). In other implementations, the sphygmomanometer 410 readings are obtained by the portable electronic device 100 by communicative coupling between the sphygmomanometer 410 and the portable electronic device 100. For example, the portable electronic device 100 has an application programming interface, or the like, that facilitates electronic data communications with the sphygmomanometer 410, and the sphygmomanometer 410 is configured to generate digital outputs for communication to the portable electronic device 100.

Based on obtaining one or more CFBF-based BPM readings and one or more arterial BPM readings, the processor(s) 130 can generate one or more calibration mappings 155. In some implementations, the processor(s) 130 generate the calibration mapping(s) 155 as one or more lookup tables that associate CFBF-based BPM readings with corresponding arterial BPM readings. With such implementations, a subsequently obtained CFBF-based BPM reading can be looked up in the lookup table to find a corresponding arterial BPM reading, if available. In other implementations, the processor(s) 130 compute the calibration mapping(s) 155 as one or more mathematical correlations (e.g., a parametric function, or the like) that formulaically relate CFBF-based BPM readings with corresponding arterial BPM readings. With such implementations, a subsequently obtained CFBF-based BPM reading can be used as an input variable to the mathematical correlation by which to compute a corresponding arterial BPM reading. In some implementations, a mathematical correlation is used to populate some or all of a lookup table. In some embodiments, separate calibration mappings 155 are generated for systolic and diastolic measurements. In other embodiments a single calibration mapping 155 is generated for both systolic and diastolic measurements.

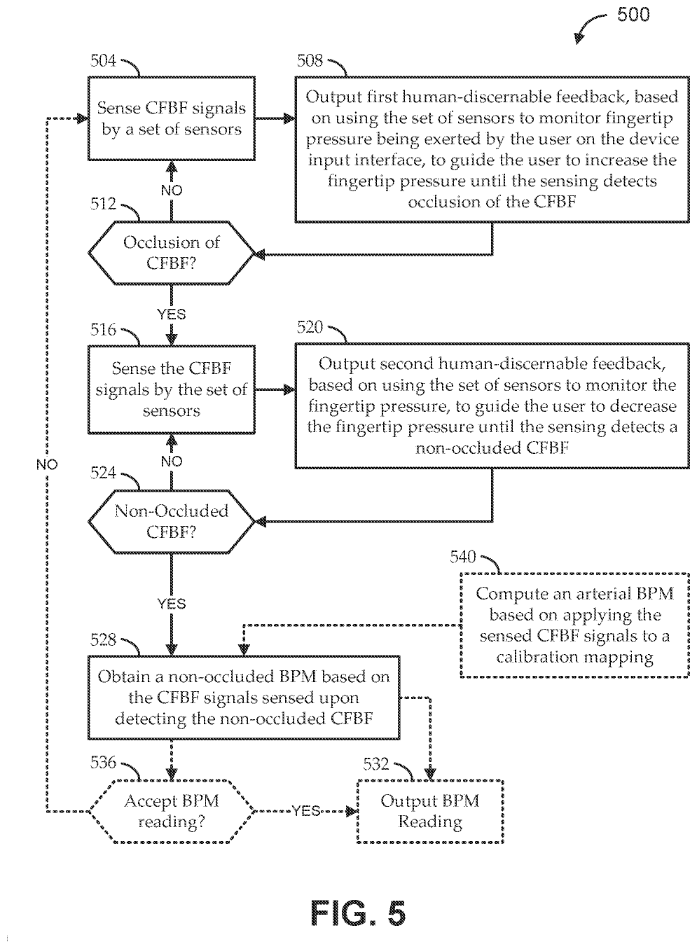

FIG. 5 shows a flow diagram of an illustrative method 500 for measuring blood pressure of a user by a portable electronic device, according to various embodiments. The portable electronic device has a device output interface and a device input interface, the device input interface having a set of sensors integrated therewith including a force sensor. Embodiments of the method 500 begin at stage 504 by first sensing capillary fingertip blood flow (CFBF) signals by the set of sensors for a fingertip by which the user is presently applying fingertip pressure to the device input interface. The CFBF signals correspond to heartbeat signals of the user. The heart pumps blood into main arteries, and the blood flows through various portions of the circulatory system until it reaches the capillaries in the fingertips of the user. As such, under normal conditions, dynamics of blood flow in the capillaries is functionally related to dynamics of blood flow in the main arteries; and measuring blood pressure based on CFBF can provide an analog to measuring blood pressure at the main arteries, or other arteries of the circulatory system (e.g., the brachial artery). In some implementations, the CFBF signals are sensed using the force sensor. In some implementations, the set of sensors further includes an optical fingerprint sensor, and the CFBF signals are sensed using the optical fingerprint sensor. For example, optical technique (e.g., multispectral and/or other techniques) can be used to image the blood fluid dynamics in the fingertip. In some implementations, the set of sensors further includes an ultrasound sensor, and the CFBF signals are sensed using the ultrasound sensor. For example, ultrasonic technique can be used to detect blood fluid dynamics in the fingertip.

At stage 508, embodiments can output, concurrent with the first sensing in stage 504, first human-discernable feedback via the at least one device output interface to guide the user to increase the fingertip pressure until the first sensing detects occlusion of the CFBF. The first human-discernable feedback is generated based on using the force sensor to monitor the fingertip pressure being exerted by the user on the device input interface. At stage 512, embodiments can determine whether the first sensing has detected occlusion of the CFBF. If not, embodiments can continue to iterate stages 504-512 until the first sensing has detected occlusion of the CFBF. For example, embodiments can instruct the user to press down harder with her finger, while measuring how hard the finger is being pressed with the force sensor, until one or more sensors detects that CFBF has become fully occluded.

When the determination at stage 512 is that the first sensing has detected occlusion of the CFBF, embodiments can proceed with obtaining a non-occluded blood pressure measurement (BPM) in stages 516-528. At stage 516, embodiments can second sense the CFBF signals by the set of sensors. In some implementations, the same one or more sensors can continue to sense the CFBF, such that the first sensing in stage 504 becomes the second sensing in stage 516. In other implementations, a first one or more of the sensors performs the first sensing in stage 504, and a different one or more sensors performs the second sensing in stage 516. At stage 520, embodiments can output, concurrent with the second sensing in stage 516, second human-discernable feedback via the at least one device output interface to guide the user to decrease the fingertip pressure until the second sensing detects a non-occluded CFBF. The second human-discernable feedback is generated based on using the force sensor to monitor the fingertip pressure being exerted by the user on the device input interface.

At stage 524, embodiments can determine whether the second sensing has detected the non-occluded CFBF. If not, embodiments can continue to iterate stages 516-524 until the second sensing has detected the non-occluded CFBF. For example, embodiments can instruct the user to release fingertip pressure, while keeping the fingertip on the device input interface, until one or more sensors can detect the non-occluded CFBF. Upon detecting the non-occluded CFBF, at stage 528, embodiments can obtain the non-occluded BPM based on the CFBF signals as sensed upon the detecting the non-occluded CFBF. In some embodiments, the non-occluded BPM obtained in stage 528 corresponds to a systolic BPM of the user (e.g., the fingertip pressure is only partially reduced until systolic CFBF begins). In other embodiments, the non-occluded BPM obtained in stage 528 corresponds to a diastolic BPM of the user (e.g., the fingertip pressure is substantially completely reduced to allow full diastolic CFBF). In other embodiments, multiple non-occluded BPMs are obtained in stage 620, such as corresponding to both a systolic BPM and a diastolic BPM of the user.

In some embodiments, the obtaining the non-occluded BPM in stages 516-528 includes obtaining at least a systolic BPM and a diastolic BPM by at least two corresponding iterations of stages 516-528. Obtaining the systolic BPM can be performed responsive to the detecting the occlusion of the CFBF in stage 512. Obtaining the systolic BPM can include outputting in the first iteration of stage 520, concurrent with the second sensing in a first iteration of stage 516, the second human-discernable feedback, based on using the force sensor to monitor the fingertip pressure, to guide the user to reduce the fingertip pressure only until the second sensing detects a systolic CFBF in a first iteration of stage 524. In a first iteration of stage 528, the systolic BPM is obtained based on the CFBF signals as sensed upon the detecting the systolic CFBF. Obtaining a diastolic BPM can be performed responsive to the detecting the systolic CFBF in the first iteration of stage 528. Obtaining a diastolic BPM can include outputting in a second iteration of stage 520, concurrent with the second sensing in a second iteration of stage 516, the second human-discernable feedback, based on using the force sensor to monitor the fingertip pressure, to guide the user slowly to further reduce the fingertip pressure until the second sensing detects a diastolic CFBF in a second iteration of stage 524. In a second iteration of stage 528, the diastolic BPM is obtained based on the CFBF signals as sensed upon the detecting the diastolic CFBF. In some such embodiments, the obtaining the systolic BPM commences automatically (e.g., without any user input, etc.) upon the detecting the occlusion of the CFBF, and the obtaining the diastolic BPM commences automatically upon the obtaining the systolic BPM.

In various embodiments, the human-discernable feedback can include graphical feedback output (e.g., alphanumeric, images, illumination of an indicator, etc.) via a display of the portable electronic device; audible feedback output (e.g., synthesized sounds and/or speech, recorded sounds and/or speech, etc.) via an audio transducer of the portable electronic device, and/or haptic feedback output (e.g., vibration, etc.) via a haptic output interface of the portable electronic device. In some embodiments, the first and second human-discernable feedback are provided in substantially the same manner, such as by using the same device output interface in the same way. For example, the first human-discernable feedback during stages 504-512 includes text and related graphics, displayed on a display screen of the portable electronic device, instructing the user to continue applying increasing fingertip pressure; and the second human-discernable feedback during stages 516-528 also includes text and related graphics, displayed on a display screen of the portable electronic device, instructing the user to slowly reduce fingertip pressure being applied. In other embodiments, the first and second human-discernable feedback are provided in substantially different manners, such as by using different device output interfaces and/or in different ways. For example, the first human-discernable feedback during stages 504-512 includes synthesized audio instruction, played through an audio transducer of the portable electronic device, instructing the user to continue applying increasing fingertip pressure; and the second human-discernable feedback during stages 516-528 includes a graphical meter, displayed on a display screen of the portable electronic device, that indicates the amount of fingertip pressure presently being applied in relation to a target level.

Having obtained the non-occluded BPM at stage 528, some embodiments can output a non-occluded BPM reading at stage 532. For example, the non-occluded BPM can be output to internal non-transient storage of the portable electronic device, to a display of the portable electronic device (e.g., via a display interface), to a peripheral device (e.g., external storage, external display, network connected devices, etc.), to another computational platform, etc. In some embodiments, the outputting at stage 532 involves outputting a quantity according to a particular unit basis. For example, human BPM readings are often communicated in units of millimeters of mercury (mmHg). In other embodiments, the outputting at stage 532 can involve comparing the obtained non-occluded BPM to one or more thresholds, levels, etc., and outputting the non-occluded BPM reading in accordance with such a comparison. For example, if the non-occluded BPM is determined to be within a determined normal range (e.g., within a threshold range of prior measurements for the user, statistical measurements across a population, etc.), the outputting at stage 532 can involve displaying the unit-based reading in a particular color (e.g., green), displaying “Your BPM is Normal!”, displaying a stoplight with the green light illuminated, displaying a color bar with the user's non-occluded BPM reading indicated as within a “normal” (e.g., green) region of the color bar, etc. Similarly, if the non-occluded BPM is determined to be outside a determined normal range (e.g., higher than a predetermined healthy level), the outputting at stage 532 can involve displaying the unit-based reading in a particular color (e.g., yellow or red), displaying “Your BPM is Too High”, displaying suggestive feedback (e.g., “Sit and relax for a bit . . . ”), displaying a stoplight with the yellow or red light illuminated, displaying a color bar with the user's non-occluded BPM reading indicated in one of the regions of the color bar outside the normal range, etc.

In embodiments that obtain the non-occluded BPM as including at least systolic BPM and diastolic BPM readings, the outputting at stage 532 can include outputting the non-occluded BPM as indicating one of the systolic BPM or the diastolic BPM, as separately indicating each of the systolic BPM and the diastolic BPM, and/or as indicating both the systolic BPM and the diastolic BPM as a combined result. As one example, each of the systolic BPM and the diastolic BPM is stored as a separate value in a memory. As another example, a combined result is displayed (e.g., “Your BPM is 120/80 mmHg”). Additionally or alternatively, the systolic BPM and the diastolic BPM can be analyzed against other levels or thresholds, separately and/or together, to provide other types of output. For example, a displayed result can indicate “Your systolic BPM is a bit high, but your diastolic BPM looks good.”

In some embodiments, the method 500 further determines, at stage 536, whether the obtaining the non-occluded BPM in stages 516-528 meets a set of predetermined acceptance criteria. In some implementations, the set of acceptance criteria includes a range of acceptable value for the non-occluded BPM; and the acceptance criteria are met if the non-occluded BPM obtained in stage 528 is within that range. In some cases, the range is selected so that a reading is rejected if indicative of a mistake, rather than possibly indicating an abnormal BPM. In other cases, the range is selected to be rejected if the non-occluded BPM deviates by more than a predetermined magnitude (e.g., number, percentage, etc.) from a determined “normal” reading (e.g., based on prior readings for the user, based on statistics from users of similar demographics, etc.), thereby indicating a mistake in the reading. In some implementations, the set of acceptance criteria includes a range of acceptable value for amount of fingertip pressure at one or more stages of the method 500, and/or for a rate of change in fingertip pressure at one or more stages of the method 500, based on force sensor measurements; and the acceptance criteria are met if the non-occluded BPM obtained in stage 528 is within that range. For example, the force sensor measurements can indicate that the user may have decreased fingertip pressure too quickly or too much during detection of CFBF occlusion and/or during obtaining of the non-occluded BPM, thereby indicating that the resulting non-occluded BPM reading is likely inaccurate. If the determination at stage 536 is that the obtaining the non-occluded BPM in stages 516-528 fails to meet the set of predetermined acceptance criteria, embodiments can repeat some or all of stages 504-528 until the obtaining the non-occluded BPM meets the set of predetermined acceptance criteria. In some embodiments, the outputting at stage 532 is performed only after determining at stage 536 that the obtaining the non-occluded BPM meets the set of predetermined acceptance criteria.

In some embodiments, obtaining the non-occluded BPM in stage 528 involves generating an arterial BPM at stage 540. As described herein, a calibration routine can be used to develop a functional relationship between one or more non-occluded BPMs obtained based on the CFBF of the user and one or more corresponding non-occluded BPMs obtained concurrently based on arterial blood flow of the user. For example, a user can obtain one or more non-occluded BPM readings based on the CFBF in accordance with stages 504-528, while concurrently obtaining one or more non-occluded BPM readings for blood flow in the user's brachial artery using a sphygmomanometer (e.g., a blood pressure cuff). As corresponding values are recorded to the portable electronic device, the device can generate a calibration mapping that include a mathematical function that relates the values, and/or includes a lookup table with corresponding values, or the like. As such, at stage 540, embodiments can compute the arterial BPM by applying the calibration mapping to the CFBF signals as sensed upon the detecting the non-occluded CFBF at stage 524, such that the obtained non-occluded BPM at stage 528 is (or includes) the computed arterial BPM.

While this disclosure contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

A recitation of “a”, “an” or “the” is intended to mean “one or more” unless specifically indicated to the contrary. Ranges may be expressed herein as from “about” one specified value, and/or to “about” another specified value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. When such a range is expressed, another embodiment includes from the one specific value and/or to the other specified value. Similarly, when values are expressed as approximations, by use of the antecedent “about,” it will be understood that the specified value forms another embodiment. It will be further understood that the endpoints of each of the ranges are included with the range.

All patents, patent applications, publications, and descriptions mentioned here are incorporated by reference in their entirety for all purposes. None is admitted to be prior art.

What is claimed is:

1. A system for measuring blood pressure of a user, the system comprising:
one or more processors configured to communicate with a device input interface of a portable electronic device and at least one device output interface of the portable electronic device, the device input interface having a set of sensors integrated therewith including a force sensor; and
a non-transient, processor-readable memory having instructions stored thereon, which, when executed, cause the one or more processors to perform steps comprising:
first sensing capillary fingertip blood flow (CFBF) signals by the set of sensors for a fingertip by which the user is presently applying fingertip pressure to the device input interface, the CFBF signals corresponding to heartbeat signals of the user;
outputting, concurrent with the first sensing, first human-discernable feedback via the at least one device output interface, based on monitoring the fingertip pressure by the force sensor, to guide the user to increase the fingertip pressure until the first sensing detects occlusion of the CFBF; and
obtaining a non-occluded blood pressure measurement (BPM), responsive to the detecting the occlusion of the CFBF, by:
second sensing the CFBF signals by the set of sensors; and
outputting, concurrent with the second sensing, second human-discernable feedback via the at least one device output interface, based on monitoring the fingertip pressure by the force sensor, to guide the user to reduce the fingertip pressure until the second sensing detects a non-occluded CFBF,
such that the non-occluded BPM is obtained based on the CFBF signals as sensed upon the detecting the non-occluded CFBF.

2. The system of claim 1, wherein the instructions, when executed, cause the one or more processors to perform steps further comprising:
outputting the non-occluded BPM via the at least one device output interface.

3. The system of claim 1, wherein:
the non-transient, processor-readable memory further has, stored thereon, a calibration mapping indicating a functional relationship, obtained by a prior calibration routine, between one or more non-occluded BPMs obtained based on the CFBF of the user and one or more corresponding non-occluded BPMs obtained concurrently based on arterial blood flow of the user; and
the non-occluded BPM is obtained as an arterial BPM computed by applying the calibration mapping to the CFBF signals as sensed upon the detecting the non-occluded CFBF.

4. The system of claim 1, wherein:
the obtaining the non-occluded BPM comprises:
obtaining a systolic BPM, responsive to the detecting the occlusion of the CFBF, by outputting, concurrent with the second sensing, the second human-discernable feedback, based on monitoring the fingertip pressure by the force sensor, to guide the user to reduce the fingertip pressure only until the second sensing detects a systolic CFBF, such that the systolic BPM is obtained based on the CFBF signals as sensed upon the detecting the systolic CFBF; and
obtaining a diastolic BPM, responsive to the detecting the systolic CFBF, by outputting, concurrent with the second sensing, the second human-discernable feedback, based on monitoring the fingertip pressure by the force sensor, to guide the user to further reduce the fingertip pressure until the second sensing detects a diastolic CFBF, such that the diastolic BPM is obtained based on the CFBF signals as sensed upon the detecting the diastolic CFBF.

5. The system of claim 4, wherein:
the obtaining the systolic BPM commences automatically upon the detecting the occlusion of the CFBF; and
the obtaining the diastolic BPM commences automatically upon the obtaining the systolic BPM.

6. The system of claim 1, wherein:
the device input interface comprises an optical fingerprint sensor of the portable electronic device, the optical fingerprint sensor having the force sensor integrated therein, as a discrete package, to monitor force exerted on a top cover layer of the optical fingerprint sensor;
the first and second sensing the CFBF signals is by the optical fingerprint sensor.

7. The system of claim 1, wherein the first and second sensing the CFBFsignals is by the force sensor.

8. The system of claim 1, wherein the device input interface is a discrete component package installed in the portable electronic device, the discrete component package comprising a physical button having the force sensor integrated therein to monitor force exerted on the physical button.

9. The system of claim 1, wherein the device input interface comprises a touchscreen interface of the portable electronic device, the touchscreen interface having the force sensor integrated therein to monitor force exerted on the touchscreen interface.

10. The system of claim 1, wherein each of the first and second human-discernable feedback comprises at least one of:
graphical feedback output via a display of the portable electronic device;
audible feedback output via an audio transducer of the portable electronic device; or
haptic feedback output via a haptic output interface of the portable electronic device.

11. A method for measuring blood pressure of a user by a portable electronic device having a device input interface and at least one device output interface, the device input interface having a set of sensors integrated therewith including a force sensor, the method comprising:
first sensing capillary fingertip blood flow (CFBF) signals by the set of sensors for a fingertip by which the user is presently applying fingertip pressure to the device input interface, the CFBF signals corresponding to heartbeat signals of the user;
outputting, concurrent with the first sensing, first human-discernable feedback via the at least one device output interface, based on monitoring the fingertip pressure by the force sensor, to guide the user to increase the fingertip pressure until the first sensing detects occlusion of the CFBF; and
obtaining a non-occluded blood pressure measurement (BPM), responsive to the detecting the occlusion of the CFBF, by:
second sensing the CFBF signals by the set of sensors; and
outputting, concurrent with the second sensing, second human-discernable feedback via the at least one device output interface, based on monitoring the fingertip pressure by the force sensor, to guide the user to reduce the fingertip pressure until the second sensing detects a non-occluded CFBF, such that the non-occluded BPM is obtained based on the CFBF signals as sensed upon the detecting the non-occluded CFBF.

12. The method of claim 11, further comprising:
determining whether the obtaining the non-occluded BPM meets a set of predetermined acceptance criteria; and
repeating the first sensing the CFBF, the outputting the first human-discernable feedback, and the obtaining the non-occluded BPM, iteratively, until the obtaining the non-occluded BPM meets the set of predetermined acceptance criteria.

13. The method of claim 11, wherein:
the non-occluded BPM is obtained as an arterial BPM computed by applying a calibration mapping to the CFBF signals as sensed upon the detecting the non-occluded CFBF, the calibration mapping corresponding to a functional relationship, obtained by a prior calibration routine, between one or more non-occluded BPMs obtained based on the CFBF of the user and one or more corresponding non-occluded BPMs obtained concurrently based on arterial blood flow of the user.

14. The method of claim 11, wherein the obtaining the non-occluded BPM comprises:
obtaining a systolic BPM, responsive to the detecting the occlusion of the CFBF, by outputting, concurrent with the second sensing, the second human-discernable feedback, based on monitoring the fingertip pressure by the force sensor, to guide the user to reduce the fingertip pressure only until the second sensing detects a systolic CFBF, such that the systolic BPM is obtained based on the CFBF signals as sensed upon the detecting the systolic CFBF; and
obtaining a diastolic BPM, responsive to the detecting the systolic CFBF, by outputting, concurrent with the second sensing, the second human-discernable feedback, based on monitoring the fingertip pressure by the force sensor, to guide the user slowly to further reduce the fingertip pressure until the second sensing detects a diastolic CFBF, such that the diastolic BPM is obtained based on the CFBF signals as sensed upon the detecting the diastolic CFBF.

15. The method of claim 14, further comprising:
outputting, via the device output interface, the non-occluded BPM as separately indicating each of the systolic BPM and the diastolic BPM.

16. The method of claim 14, wherein:
the obtaining the systolic BPM commences automatically upon the detecting the occlusion of the CFBF; and
the obtaining the diastolic BPM commences automatically upon the obtaining the systolic BPM.

17. The method of claim 11, wherein the first and second sensing the CFBF signals is by the force sensor.

18. The method of claim 11, wherein:
the set of sensors further includes an optical fingerprint sensor; and
the first and second sensing the CFBF signals is by the optical fingerprint sensor.

19. The method of claim 11, wherein each of the first and second human-discernable feedback comprises at least one of:
graphical feedback output via a display of the portable electronic device;
audible feedback output via an audio transducer of the portable electronic device; or
haptic feedback output via a haptic output interface of the portable electronic device.

20. An electronic device comprising a system for measuring blood pressure of a user, wherein the system comprising:
one or more processors configured to communicate with a device input interface of a portable electronic device and at least one device output interface of the portable electronic device, the device input interface having a set of sensors integrated therewith including a force sensor; and
a non-transient, processor-readable memory having instructions stored thereon, which, when executed, cause the one or more processors to perform steps comprising:
first sensing capillary fingertip blood flow (CFBF) signals by the set of sensors for a fingertip by which the user is presently applying fingertip pressure to the device input interface, the CFBF signals corresponding to heartbeat signals of the user;
outputting, concurrent with the first sensing, first human-discernable feedback via the at least one device output interface, based on monitoring the fingertip pressure by the force sensor, to guide the user to increase the fingertip pressure until the first sensing detects occlusion of the CFBF; and
obtaining a non-occluded blood pressure measurement (BPM), responsive to the detecting the occlusion of the CFBF, by:
second sensing the CFBF signals by the set of sensors; and
outputting, concurrent with the second sensing, second human-discernable feedback via the at least one device output interface, based on monitoring the fingertip pressure by the force sensor, to guide the user to reduce the fingertip pressure until the second sensing detects a non-occluded CFBF,
such that the non-occluded BPM is obtained based on the CFBF signals as sensed upon the detecting the non-occluded CFBF.
